(19) United States
(12) Patent Application Publication
(10) Pub. No.: US 2010/0004553 A1
(43) Pub. Date: Jan. 7, 2010

(54) Device for Eliminating Liquid Condensation in a Mouthpiece

(75) Inventors: Hans-Olov Moberg, Bro (SE); Lars-Ake Lejon, Uppsala (SE)

Correspondence Address: POTOMAC PATENT GROUP PLLC, P. O. BOX 270, FREDERICKSBURG, VA, 22404, US

(73) Assignee: NANOPULS AB, Uppsala (SE)

(21) Appl. No.: 12/513,880

(22) PCT Filed: Dec. 4, 2007
(86) PCT No.: PCT/SE2007/001078

§ 371 Date: May 16, 2009

(30) Foreign Application Priority Data
Dec. 11, 2006 (SE) 0602646-2

(51) Int. Cl. A61B 5/08

(52) U.S. Cl. 600/529

(57) ABSTRACT

In a device for elimination of liquid condensation in a mouthpiece intended to be used in the analysis of exhalation air a tube shaped body of a heat conducting material is provided with heating means and is circumvented by a mouthpiece preferably for one time use.

The present invention refers to a device for eliminating liquid condensation in a mouthpiece used in the analysis of exhalation air.

In the analysis of exhalation air a problem consists in the fact that the exhalation air is supplied to an analysis instrument via a mouthpiece having a hose connected to it, whereby the mouthpiece, especially if the device is used outdoors at low temperatures, often is having a low temperature low enough to generate liquid condensation in the mouthpiece, the condensation deteriorating the overall sensitivity of the instrument and especially being a big problem if one wishes to measure the content of water in the exhalation air which e.g. is the case at the device according to the Swedish patent application 0600724-9 “A method and a device for measuring breath temperature in breath alcohol testing ”. It has turned out to be very difficult to prevent that the temperature of the exhalation air on its way to the mouth-piece does not drop to such low levels that condensation appears while meeting the hygienic demands required.

It is an object of the present invention to provide a device in a mouthpiece intended to be used for the analysis of exhalation air where the temperature of the exhalation air does not drop to a temperature low enough to generate condensation.

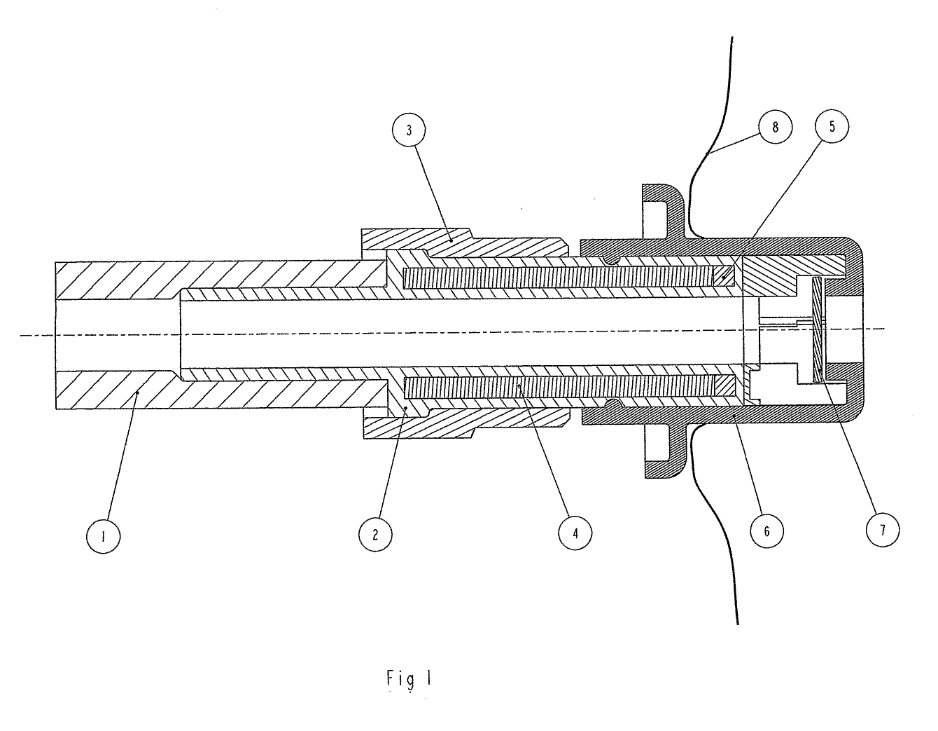

The invention will now be described in detail, reference being made to the enclosed drawing in which FIG. 1 shows an embodiment of a device according to the invention.

In FIG. 1 reference 1 denotes a hose which in a way known per se connects a mouthpiece to an analysis device not shown in the FIGURE. Reference 2 denotes a tube shaped body manufactured from a heat conducting material having a high heat capacity. The tube shaped body is connected to the hose via a housing 3 and is provided with an inner heating element 4 heated from a supply not shown in the FIGURE in a way known per se and further being provided with a temperature sensor 5, which controls the heat of the heating element and the heat conducting material in such a way that the material is kept at a suitable temperature in the order of 45° C. The device further comprises a mouthpiece 6 suitably manufactured from plastic and intended for one-time use. The mouthpiece 6 is shaped so as to be easily snapped on the tube shaped body and thus having a big contact surface against the body. The device shown is—when in use—inserted in a mouth cavity 8 and is furthermore provided with a non-return valve 7 so as to prevent air from passing into the mouth cavity.

In the device shown the mouthpiece 6 will thus circumvent the heated body 2 and have a big connecting surface against the body which means that heating will take place well into the mouth cavity and a non-interrupted heating chain will be obtained through the mouth-piece to the hose 1 which in a way known per se is also provided with a heating equipment not shown. In the invention it is also essential that the mouthpiece itself 6 is intended for one-time use and easily can be applied on the pre-heated body, whereby contamination between different users is prevented.

In the device shown a non-interrupted heating chain between the mouth cavity and the analysis instrument is obtained in a simple and hygienic way so as to considerably increase the sensitivity of the analysis instrument in use.

1.-5. (canceled)

6. A device for eliminating liquid condensation in a mouthpiece for analysis of exhalation air, comprising a tube-shaped body made from a heat conducting material and having a heater and configured to be inserted into a mouth cavity, whereby the mouthpiece is applied so as to enfold the tube-shaped body.

7. The device of claim 6, wherein the heater is enclosed in a wall of the tube-shaped body.

8. The device of claim 7, wherein a temperature of the tube-shaped body is controlled by a temperature sensor arranged in the wall.

9. The device of claim 6, wherein the mouthpiece is configured for one-time use.

10. The device of claim 9, wherein the mouthpiece protrudes beyond the tube-shaped body into the mouth cavity.
